(19) United States
(12) Patent Application Publication
(10) Pub. No.: US 2022/0241164 A1
(43) Pub. Date: Aug. 4, 2022

(54) Oral Care Compositions

(71) Applicant: Colgate-Palmolive Company, New York, NY (US)

(72) Inventors: Payal ARORA, Bridgewater, NJ (US); Harsh Mahendra TRIVEDI, Hillsborough, NJ (US); Zhigang HAO, Bridgewater, NJ (US); Paul THOMSON, Piscataway, NJ (US); Manish MANDHARE, Navi Mumbai (IN); Yu WANG, Edison, NJ (US); Chi-Yuan CHENG, Hillsborough, NJ (US); Long PAN, Somerset, NJ (US)

(73) Assignee: Colgate-Palmolive Company, New York, NY (US)

(21) Appl. No.: 17/250,562

(22) PCT Filed: Dec. 17, 2020
(86) PCT No.: PCT/US2020/070921

§ 371 Date: Feb. 3, 2021

(30) Foreign Application Priority Data
Dec. 26, 2019 (IN) 201911053977

(51) Int. Cl. A61K 8/35; A61Q 11/00; A61K 8/46

(52) U.S. Cl. CPC A61K 8/35; A61K 8/463; A61Q 11/00

(57) ABSTRACT

Described herein are—inter alia—complexes comprising a curcuminoid; and an anionic surfactant. Methods of making and using same are also described.

### BACKGROUND

Periodontal disease is an inflammatory disorder of the supporting tissue of the teeth and one of the most difficult problems in dentistry. It results from initial colonization by keystone pathogens, such as Porphyromonas gingivalis, which leads to a bacterial dysbiosis, and inflammatory response. This inflammation ultimately leads to bone loss and tooth loss that is characteristic of periodontal disease. In fact, periodontal disease is the major cause of tooth loss in adults over 35 years of age. The two most common periodontal diseases are chronic gingivitis and chronic destructive periodontitis.

Gingivitis is an inflammation of the gingiva, characterized by swelling, redness, changes in normal contours, watery exudate, and bleeding. It is often caused by the infection of the gingiva by bacterial plaque, calculus, and/or mechanical injury. Left untreated, gingivitis usually results in the formation of periodontal pockets, wherein the bacteria in the plaque attack the periodontium (periodontal tissues supporting the teeth), causing a chronic inflammation of the periodontium known as periodontitis or pyorrhea. In advanced stages of periodontitis, the bacteria eventually erode the bone surrounding the teeth, often resulting in the loss of the affected teeth.

Current therapies for gingivitis and/or periodontitis include improved oral hygiene to eliminate supragingival plaque and calculus, professional prophylaxis to eliminate subgingival calculus, and/or surgery. Curcumin (diferuloylmethane) is a naturally occurring compound obtained from the rhizomes of Curcuma longa plants. It is a major component of turmeric and commonly used as a spice (curry) and coloring agent for foods, drugs, and cosmetics.

While compositions containing curcuminoids may generally be known, and the numerous benefits of curcuminoids are undeniable, curcuminoid-containing compositions often suffer from stability issues, which result in a lack of efficacy and pose challenges in manufacturing scale-up. Heretofore, attempts to overcome these stability issues have been unsuccessful. As such, there remains a need for stable oral care compositions containing a curcuminoid, that do not present manufacturing challenges.

Embodiments of the present invention are designed to address these, and other, needs.

### BRIEF SUMMARY

In some embodiments, the present invention provides an oral care composition comprising: an orally acceptable carrier; and a complex comprising: a curcuminoid; and an anionic surfactant.

Other embodiments of the present invention provide a conjugate comprising: a curcuminoid; and an alkyl sulfate surfactant; wherein the conjugate has a diffusion coefficient less than the diffusion coefficient of the curcuminoid alone.

While other embodiments of the present invention provide an oral care composition comprising: an orally acceptable carrier; a pre-mix comprising: a curcuminoid; an alkyl sulfate surfactant; and a humectant (e.g., a polyol humectant).

Yet other embodiments of the present invention provide a method of treating, preventing, or ameliorating a symptom associated with, an inflammatory disease, disorder or condition of the oral cavity comprising administering: a) any one of the oral care compositions described herein; b) any one of the conjugates described herein; or c) any one of the complexes described herein, to the oral cavity of a subject in need thereof.

Further embodiments of the present invention provide a method for treating, preventing, or ameliorating a symptom associated with, a viral infection comprising administering: a) any one of the oral care compositions described herein; b) any one of the conjugates described herein; or c) any one of the complexes described herein, to the oral cavity of a subject in need thereof.

### BRIEF DESCRIPTION OF THE DRAWINGS

FIG. 1 depicts the results of the liquid chromatography-mass spectrometry (LCMS) analysis.

FIG. 2 depicts data demonstrating the impact that exemplary curcuminoids of the present invention (curcumin [dashed line] and tetrahydrocurcumin [solid line]) have on PGE2.

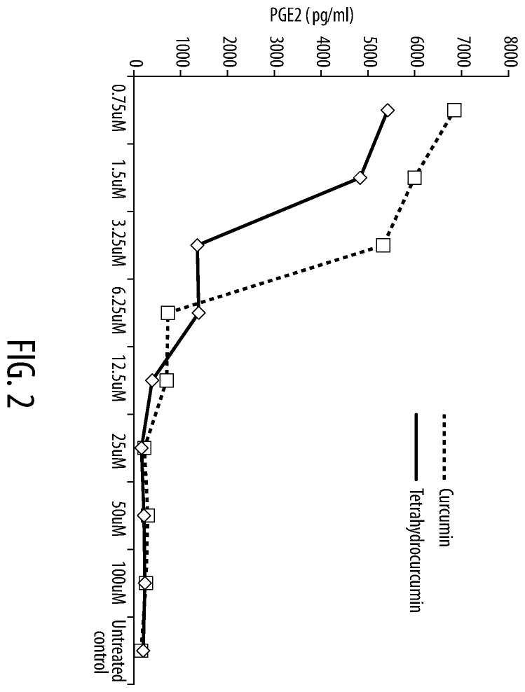

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other apparatuses and methods. Before explaining the disclosed embodiments of the present invention in detail, it is to be understood that the invention is not limited in its application to the details of any particular embodiment shown. The terminology used herein is for the purpose of description and not of limitation.

As used herein and in the appended claims, the singular forms “a”, “an”, and “the” include plural references unless the context dictates otherwise. The singular form of any class of the ingredients refers not only to one chemical species within that class, but also to a mixture of those chemical species; for example, the term “vitamin D” in the singular form, may refer to a mixture of compounds each of which is also considered a vitamin D. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

The abbreviations and symbols as used herein, unless indicated otherwise, take their ordinary meaning. The abbreviation “wt %” means percent by weight. The symbol “μL,” refers to a microliter, or 10⁻⁶ liters. The symbol “°” refers to a degree, including a degree of an angle and degree of Celsius.

When referring to chemical structures, and names, the symbols “C”, “H”, and “O” mean carbon, hydrogen, and oxygen, respectively. The symbols “—” and “═” mean single bond, and double bond, respectively.

The abbreviations “dy”, “mo”, “ppm”, “PBS”, “c-DNA”, “RNA”, “qPCR”, “GAPDH”, “USP”, “EP”, “FD&C”, “pH” mean “days”, “months”, “parts per million”, “phosphate-buffered saline” “complementary deoxyribonucleic acid”, “ribonucleic acid”, “quantitative polymerase chain reaction”, “glyceraldehyde 3-phosphate dehydrogenase”, “United States Pharmacopeia”, “European Pharmacopeia”, “Food, Drug & Cosmetics”, negative logarithm of the molar concentration of hydronium ions, respectively.

The term “about” when referring to a number means any number within a range of 10% of the number. For example, the phrase “about 0.050 wt %” refers to a number between and including 0.04500 wt % and 0.05500 wt %.

As used throughout, ranges are used as shorthand for describing each and every value that is within the range. Any value within the range can be selected as the terminus of the range.

The term “mixture” is to be interpreted broadly. It refers to a mixture of ingredients. The mixture may be a solid, liquid, semisolid. If a mixture is a liquid, a mixture may be a solution, an emulsion, a dispersion, a mixture displaying the Tyndall effect, or any other homogeneous mixture. Under one embodiment, the mixture is shelf stable. When referring to a list of ingredients, unless specifically indicated otherwise, the term “mixture” refers to a mixture of the aforementioned ingredients with each other, a mixture of any of aforementioned ingredients with other ingredients that are not aforementioned, and to a mixture of several aforementioned ingredients with other ingredients that are not aforementioned. For example, the term “mixture” in the phrase “the fluoride source is selected from the group consisting of stannous fluoride, sodium fluoride, amine fluoride, sodium monofluorophosphate, and mixtures thereof” refers to any of the following: a mixture of stannous fluoride and sodium fluoride; or a mixture of stannous fluoride and amine fluoride; or a mixture of stannous fluoride and sodium monofluorophosphate; or a mixture of sodium fluoride and amine fluoride; or a mixture of sodium fluoride and sodium monofluorophosphate; or a mixture of amine fluoride, sodium monofluorophosphate; or a mixture of stannous fluoride and any other fluoride source; or a mixture of sodium fluoride and any other fluoride source; or a mixture of amine fluoride and any other fluoride source; or a mixture of sodium monofluorophosphate and any other fluoride source, and other combinations thereof.

Any member in a list of species that are used to exemplify or define a genus may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All references cited herein are hereby incorporated by reference in their entireties. In the event of a conflict in a definition in the present disclosure and that of a cited reference, the present disclosure controls.

In other embodiments, the present invention provides an oral care composition comprising: an orally acceptable carrier; and a complex comprising: a curcuminoid; and an anionic surfactant.

In some embodiments, the curcuminoid is selected from: curcumin (tetrahydrodiferuloylmethane); a curcumin derivative; a curcumin analogue; and a preparation of the plant Curcuma longa or another curcumin-containing plant. In further embodiments, the curcuminoid is selected from: curcumin; turmeric; phydroxycinnamoyl(feruloyl)methane, p,p′-dihydroxydicinnamoylmethane, desmethoxycurcumin, bis-desmethoxycurcumin, sodium curcuminate, diacetylcurcumin, triethylcurcumin, and tetrahydrocurcumin. In certain embodiments, the curcuminoid comprises tetrahydrocurcumin.

In other embodiments, the oral care composition comprises from about 0.001 wt. % to about 10 wt. %, of a curcuminoid, optionally 0.01 wt. % to about 7.5 wt. %, or 0.05 wt. % to about 5 wt. %, or about 0.1 wt. % to about 2.5 wt. %, or about 0.15 wt. % to about 2 wt. %, or about 0.2 wt. % to about 1 wt. %, or about 0.25 wt. % to about 0.5 wt. %, or about 0.3 wt. %, based on the total weight of the oral care composition. The oral care composition according to any foregoing claim, comprising about 0.01 wt. %, 0.02 wt. %, about 0.025 wt. %, about 0.03 wt. %, about 0.04 wt. %, about 0.05 wt. %, about 0.06 wt. %, about 0.07 wt. %, about 0.075 wt. %, about 0.08 wt. %, about 0.09 wt. %, about 0.1 wt. %, about 0.2 wt. %, about 0.3 wt. %, about 0.4 wt. %, or about 0.5 wt. %, of a curcuminoid, based on the total weight of the oral care composition.

In some embodiments, the anionic surfactant comprises an alkyl sulfate surfactant. In further embodiments, the alkyl sulfate surfactant is selected from: sodium lauryl sulfate; sodium laureth sulfate; and a combination of thereof. In still further embodiments, the anionic surfactant comprises from about 0.01 wt. % to about 5 wt. %, optionally from about 0.05 wt. % to about 4 wt. %, or from about 0.1 wt. % to about 3 wt. %, or about 0.5 wt. % to about 2.5 wt. %, or about 0.75 wt. % to about 2 wt. %, or about 1 wt. % to about 1.75 wt. %, or about 1.5 wt. %, based on the total weight of the oral care composition.

Yet other embodiments provide oral care compositions comprising a curcuminoid; an anionic surfactant; and a polyol humectant. In some embodiments, the polyol humectant is selected from sorbitol; glycerin; and a combination thereof. Still further embodiments provide oral care compositions wherein the polyol humectant comprise sorbitol.

In some embodiments, the oral care composition further comprises an abrasive system comprising a calcium abrasive, a silica abrasive or a combination thereof.

In other embodiments, the curcuminoid and the anionic surfactant are present in the complex at a molar ratio of from about 3:1 to about 1:3. In further embodiments, the curcuminoid and the anionic surfactant are present in the complex at a molar ratio of from about 3:2 to about 2:3. Still further embodiments provide compositions wherein the curcuminoid and the anionic surfactant are present in the complex at a molar ratio of about 1:1.

In certain embodiments, at least about 50% of the curcuminoid is recovered after 14 days at 60° C. In other embodiments, at least about 55% of the curcuminoid is recovered after 14 days at 60° C. In further embodiments, at least about 60% of the curcuminoid is recovered after 14 days at 60° C. In still further embodiments, at least about 65% of the curcuminoid is recovered after 14 days at 60° C. In certain embodiments, at least about 70% of the curcuminoid is recovered after 14 days at 60° C. In other embodiments, at least about 75% of the curcuminoid is recovered after 14 days at 60° C. In further embodiments, at least about 80% of the curcuminoid is recovered after 14 days at 60° C. In still further embodiments, at least about 85% of the curcuminoid is recovered after 14 days at 60° C. In other embodiments, at least about 90%, 91%, 92%, 93%, 94% or 95%, of the curcuminoid is recovered after 14 days at 60° C.

In some embodiments, the complex has a molecular weight of from about 625 to about 680. In other embodiments, the complex has a molecular weight of from about 630 to about 675. In further embodiments, the complex has a molecular weight of from about 635 to about 670. In other embodiments, the complex has a molecular weight of from about 637 to about 666. In some embodiments, the complex has a molecular weight of from 637.30 to 665.33. In certain embodiments, the complex has a molecular weight of 637.30. In other embodiments, the complex has a molecular weight of 665.33.

Yet other embodiments provide a conjugate comprising: a curcuminoid; and an alkyl sulfate surfactant; wherein the conjugate has a diffusion coefficient less than the diffusion coefficient of the curcuminoid alone. In some embodiments, the conjugate comprises a curcuminoid, wherein the curcuminoid comprises tetrahydrocurcumin. In some embodiments, the conjugate comprises an alkyl sulfate surfactant selected from sodium lauryl sulfate; sodium laureth sulfate; and a combination thereof. In some embodiments, the conjugate comprises tetrahydrocurcumin and sodium lauryl sulfate.

Some embodiments of the present invention provide a conjugate wherein the curcuminoid and the alkyl sulfate surfactant are present at a molar ratio of from about 3:1 to about 1:3. Other embodiments of the present invention provide a conjugate wherein the curcuminoid and the alkyl sulfate surfactant are present at a molar ratio of from about 3:2 to about 2:3. Further embodiments of the present invention provide a conjugate wherein the curcuminoid and the alkyl sulfate surfactant are present at a molar ratio of 1:1.

Still further embodiments provide a method of treating, preventing, or ameliorating a symptom associated with, an inflammatory disease, disorder or condition of the oral cavity comprising administering any one of the oral care compositions described herein, or any one of the conjugates described herein, to the oral cavity of a subject in need thereof.

In some embodiments, the present invention provides an oral care composition comprising: an orally acceptable carrier; a pre-mix comprising: a curcuminoid; a surfactant; and a polyol humectant. In other embodiments, the pre-mix comprises: from about 0.01 wt. % to about 1 wt. % of the curcuminoid; from about 0.5 wt. % to about 5 wt. % of the surfactant; and from about 1 wt. % to about 10 wt. % of the polyol humectant. In certain embodiments, the pre-mix comprises: from about 0.1 wt. % to about 0.5 wt. % of the curcuminoid; from about 1 wt. % to about 2 wt. % of the surfactant; and from about 2.5 wt. % to about 5 wt. % of the polyol humectant. Still further embodiments provide a pre-mix comprising: about 0.3 wt. % of the curcuminoid; about 1.5 wt. % of the surfactant; and about 4 wt. % of the polyol humectant.

In some embodiments, the pre-mix comprises a surfactant selected from: an anionic surfactant; a non-ionic surfactant; a cationic surfactant; an amphoteric surfactant; and a combination of two or more thereof. In other embodiments, the pre-mix comprises a surfactant selected from: a natural surfactant; a surfactant derived from a natural source; and a combination thereof. Still further embodiments of the present invention provide oral care compositions wherein wherein the pre-mix comprise a surfactant selected from: sodium cocoyl glutamate; lauryl glucoside; sodium methyl cocoyl taurate; sodium lauroyl sarcosinate; sodium lauryl sulfate; sodium laureth sulfate; cocamidopropyl betaine; sodium cocoamphoacetate; sodium lauryl glucose carboxylate; and a combination of two or more thereof.

In some embodiments, the oral care composition further comprises an anti-malodor agent. In some embodiments, the additional anti-malodor compound is a known odor-controlling agent. In addition, other metal-containing compounds, such as those of copper, stannous, bismuth, strontium; and succulents or other ingredients which increase salivary flow, act to wash away odors, are useful in the compositions described herein. Certain strong citrus-based flavorants, odor-absorption complexes, which entrap or adsorb malodor molecules are also useful in the claimed compositions. For example, Ordenone® has the ability to encapsulate malodor molecules such as mercaptans, sulfides and amines within its structure, as disclosed in, for example, U.S. Pat. No. 6,664,254. Odor-controlling actives suitable also include, but are not limited to, enzymes that can interrupt the process by which odors are created. For example, odor-blocking enzymes such as arginine deiminase, can be effectively formulated in the compositions of the invention. Also, molecules that effectively inhibit the bacterial production of malodor molecules can be used to control odor, for example agents that interfere with the bacterial enzymes cysteine desulfhydrase and/or methionine gamma-lyase. Odor-controlling actives suitable for odor blocking or as odor blockers, include but are not limited to agents that act by oxidizing or otherwise chemically reacting with malodor molecules, including peroxides, perchlorites, and reactive molecules with activated double bonds.

In some embodiments, the oral care compositions of the present invention comprise a carrier. The carrier may include, but is not limited to water or other aqueous solvent systems. In some embodiments, the carrier is an orally acceptable carrier. In some embodiments, the orally acceptable carrier may further comprise a humectant. Possible humectants are ethanol, a polyhydric alcohol, which includes, but is not limited to glycerin, glycol, inositol, maltitol, mannitol, sorbitol, xylitol, propylene glycol, polypropylene glycol (PPG), polyethylene glycol (PEG) and mixtures thereof, or a saccharide, which includes, but is not limited to fructose, glucose, sucrose and mixtures of saccharides (e.g. honey).

In further embodiments, the oral care composition may further comprise an anti-bacterial agent. In other embodiments, the anti-bacterial agent is selected from: triclosan (5-chloro-2-(2,4-dichlorophenoxy)phenol); 8-hydroxyquinoline and salts thereof, a zinc or stannous ion source, such as zinc citrate, zinc sulphate, zinc glycinate, sodium zinc citrate, stannous fluoride, stannous monofluorophosphate and stannous pyrophosphate; copper (II) compounds such as copper (II) chloride, fluoride, sulfate and hydroxide; phthalic acid and salts thereof such as magnesium monopotassium phthalate; sanguinarine; quaternary ammonium compounds, such as alkylpyridinium chlorides (e.g., cetylpyridinium chloride (CPC), combinations of CPC with zinc and/or enzymes, tetradecylpyridinium chloride, and N-tetradecyl-4-ethylpyridinium chloride,); bisguanides, such as chlorhexidine digluconate, hexetidine, octenidine, alexidine; halogenated bisphenolic compounds, such as 2,2′ methylenebis-(4-chloro-6-bromophenol); benzalkonium chloride; salicylanilide, domiphen bromide; iodine; sulfonamides; bisbiguanides; phenolics; piperidino derivatives such as delmopinol and octapinol; magnolia extract; thymol; eugenol; menthol; geraniol; carvacrol; citral; eucalyptol; catechol; 4-allylcatechol; hexyl resorcinol; methyl salicylate; antibiotics such as augmentin, amoxicillin, tetracycline, doxycycline, minocycline, metronidazole, neomycin, kanamycin and clindamycin; or mixtures thereof.

In some embodiments, the anti-bacterial agent is present at a concentration of from about 0.001% to about 3%, by weight, from about 0.05% to about 2%, by weight or from about 0.075% to about 1.5% by weight.

In some embodiments, the oral care composition may further include anti-caries agents, desensitizing agents, viscosity modifiers, diluents, surfactants, emulsifiers, foam modulators, pH modifying agents, abrasives, mouth feel agents, sweetening agents, flavor agents, colorants, preservatives, amino acids, anti-oxidants. anti-calculus agents, a source of fluoride ions, thickeners, an active agent for prevention or treatment of a condition or disorder of hard or soft tissue of the oral cavity, adhesive agents, a whitening agent and combinations thereof. It is understood that while general attributes of each of the above categories of materials may differ, there may be some common attributes and any given material may serve multiple purposes within two or more of such categories of materials. Preferably, the carrier is selected for compatibility with other ingredients of the composition.

Some embodiments of the present invention optionally comprise an amino acid. Suitable amino acids include, but are not limited to arginine, cysteine, leucine, isoleucine, lysine, alanine, asparagine, aspartate, phenylalanine, glutamate, glutamic acid, threonine, glutamine, tryptophan, glycine, valine, praline, serine, tyrosine, and histidine, and a combination of two or more thereof. The amino acids can include R- and L-forms and salt forms thereof. The amino acids (and salt forms thereof) can also include acid ester and/or fatty amide derivatives of the amino acid (e.g. ethyl lauroyl arginate hydrochloride (ELAH)).

Yet other embodiments of the present invention provide oral care compositions comprising an antioxidant. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

Still further embodiments of the present invention comprise an anticalculus (tartar control) agent. Suitable anticalculus agents include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. In some embodiments, the anticalculus agent is present in an amount of from about 0.1% to about 30%. The oral composition may include a mixture of different anticalculus agents. In one preferred embodiment, tetrasodium pyrophosphate (TSPP) and sodium tripolyphosphate (STPP) are used. In some embodiments, the anticalculus agent comprises TSPP at about 0.1% to about 5%, by weight. In other embodiments, the anticalculus agent comprises STPP at about 0.1% to about 10%, by weight.

Certain embodiments of the present invention comprise at least one orally acceptable source of fluoride ions. Any known or to be developed in the art may be used. Suitable sources of fluoride ions include fluoride, stannous fluoride, sodium fluoride, potassium fluoride, amine fluoride, ammonium fluoride, stannous monofluorophosphate, sodium monofluorophosphate, potassium monofluorophosphate, amine monofluorophosphate, ammonium monofluorophosphate, stannous fluorosilicate, sodium fluorosilicate, potassium fluorosilicate, amine fluorosilicate ammonium fluorosilicate, and mixtures thereof. One or more fluoride ion-releasing compound is optionally present in an amount providing a total of about 100 to about 20,000 ppm, about 200 to about 5,000 ppm, or about 500 to about 2,500 ppm, fluoride ions.

Further embodiments of the present invention comprise various dentifrice ingredients to adjust the rheology and feel of the composition such as surface active agents, thickening or gelling agents, etc.

Some embodiments of the present invention provide oral care compositions comprising a stannous ion or a stannous ion source. Suitable stannous ion sources include without limitation stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide and the like. One or more stannous ion sources are optionally and illustratively present in a total amount of about 0.01% to about 10%, for example about 0.1% to about 7% or about 1% to about 5%.

Some embodiments of the present invention provide an oral care composition comprising a surface active agent (surfactant). Suitable surfactants include without limitation water-soluble salts of C8-C20 alkyl sulfates, sulfonated monoglycerides of C8-C20 fatty acids, sarcosinates, taurates, sodium lauryl sulfate, sodium cocoyl monoglyceride sulfonate, sodium lauryl sarcosinate, sodium lauryl isoethionate, sodium laureth carboxylate and sodium dodecyl benzenesulfonate, and cocoamidopropyl betaine.

In some embodiments, the oral care composition comprises a thickening agent. Any orally acceptable thickening agent can be used, including without limitation carbomers, also known as carboxyvinyl polymers, carrageenans, also known as Irish moss and more particularly -carrageenan (iota-carrageenan), high molecular weight polyethylene glycols (such as Carbowax®, available from The Dow Chemical Company), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, and colloidal and/or fumed silica and mixtures of the same. One or more thickening agents are optionally present in a total amount of about 0.1% to about 90%, for example about 1% to about 50% or about 5% to about 35%.

Other embodiments of the present invention optionally comprise a flavorant, a sweetener, a colorant, a foam modulator, a mouth-feel agent and/or others additively may be included if desired, in the composition.

Yet other embodiments of the present invention comprise one or more additional active material(s), which is operable for the prevention or treatment of a condition or disorder of hard or soft tissue of the oral cavity, the prevention or treatment of a physiological disorder or condition, or to provide a cosmetic benefit. Examples of such further active ingredient comprise a sialagogue or saliva-stimulating agent, an antiplaque agent, an anti-inflammatory agent, and/or a desensitizing agent.

Adhesion enhancing agents can also be added to the oral care compositions which include but is not limited to waxes, inclusive of bees' wax, mineral oil, plastigel, (a blend of mineral oil and polyethylene), petrolatum, white petrolatum, shellac, versagel (blend of liquid paraffin, butene/ethylene/styrene hydrogenated copolymer) polyethylene waxes, microcrystalline waxes, polyisobutene, polyvinyl pyrrolidone/vinyl acetate copolymers, and insoluble polyacrylate copolymers.

Also effective as adhesion enhancing agents are liquid hydrophilic polymers including polyethylene glycols, nonionic polymers of ethylene oxide having the general formula: HOCH2 (CH2OCH2)n1CH2OH wherein n1 represents the average number of oxyethylene groups. Polyethylene glycols available from Dow Chemical are designated by a number such as 200, 300, 400, 600, 2000 which represents the approximate average molecular weight of the polymer, as well as nonionic block copolymer of ethylene oxide and propylene oxide of the formula: HO(C2H4O)a1(C3H6O)b1(C2H4O)c1H. The block copolymer is preferably chosen (with respect to a1, b1 and c1) such that the ethylene oxide constituent comprises from about 65 to about 75% by weight, of the copolymer molecule and the copolymer has an average molecular weight of from about 2,000 to about 15,000 with the copolymer being present in the liquid tooth whitening composition in such concentration that the composition is liquid at room temperatures.

A particularly desirable block copolymer for use in the practice of the present invention is available commercially from BASF and designated Pluraflo L1220 (PEG/PPG 116/66)which has an average molecular weight of about 9,800. The hydrophilic poly(ethylene oxide) block averages about 65% by weight of the polymer.

Synthetic anionic polycarboxylates may also be used in the oral compositions of the present invention as an efficacy enhancing agent for any antibacterial, antitartar or other active agent within the dentifrice composition. Such anionic polycarboxylates are generally employed in the form of their free acids or preferably partially or more preferably fully neutralized water soluble alkali metal (e.g. potassium and preferably sodium) or ammonium salts. Preferred are 1:4 to 4:1 copolymers of maleic anhydride or acid with another polymerizable ethylenically unsaturated monomer, preferably methylvinylether/maleic anhydride having a molecular weight (M.W.) of about 30,000 to about 1,800,000 most preferably about 30,000 to about 700,000. Examples of these copolymers are available from GAF Corporation under the trade name GANTREZ® (methylvinylether/maleic anhydride), e.g., AN 139 (M.W. 500,000), AN 119 (M.W. 250,000); S-97 Pharmaceutical Grade (M.W. 700,000), AN 169 (M.W. 1,200,000-1,800,000), and AN 179 (M.W. above 1,800,000); wherein the preferred copolymer is S-97 Pharmaceutical Grade (M.W. 700,000).

When present, the anionic polycarboxylates is employed in amounts effective to achieve the desired enhancement of the efficacy of any antibacterial, antitartar or other active agent within the oral composition. Generally, the anionic polycarboxylates is present within the oral composition from about 0.05% to about 4% by weight, preferably from about 0.5% to about 2.5% by weight.

Adhesion enhancing agents employed in compositions of various embodiments of the invention are present in an amount of from about 0 to about 20% by weight. Preferably, the adhesion enhancing agents are present in an amount of from about 2 to about 15% by weight.

Some embodiments of the present invention optionally comprise a whitening agent which includes, but is not limited to peroxide compounds such as hydrogen peroxide, peroxides of alkali and alkaline earth metals, organic peroxy compounds, peroxy acids, pharmaceutically-acceptable salts thereof, and mixtures thereof. Peroxides of alkali and alkaline earth metals include lithium peroxide, potassium peroxide, sodium peroxide, magnesium peroxide, calcium peroxide, barium peroxide, and mixtures thereof. Organic peroxy compounds include carbamide peroxide (also known as urea hydrogen peroxide), glyceryl hydrogen peroxide, alkyl hydrogen peroxides, dialkyl peroxides, alkyl peroxy acids, peroxy esters, diacyl peroxides, benzoyl peroxide, and monoperoxyphthalate, and mixtures thereof. Peroxy acids and their salts include organic peroxy acids such as alkyl peroxy acids, and monoperoxyphthalate and mixtures thereof, as well as inorganic peroxy acid salts such as persulfate, dipersulfate, percarbonate, perphosphate, perborate and persilicate salts of alkali and alkaline earth metals such as lithium, potassium, sodium, magnesium, calcium and barium, and mixtures thereof. In various embodiments, the peroxide compound comprises hydrogen peroxide, urea peroxide, sodium percarbonate and mixtures thereof.

In some embodiments, a non-peroxide whitening agent may be included in the compositions of the present invention. Whitening agents among those useful herein include non-peroxy compounds, such as chlorine dioxide, chlorites and hypochlorites. Chlorites and hypochlorites include those of alkali and alkaline earth metals such as lithium, potassium, sodium, magnesium, calcium and barium. Non-peroxide whitening agents also include colorants, such as titanium dioxide and hydroxyapatite, pigments or dyes. In some embodiments the whitening agent is separated from the aqueous carrier. In some embodiments the whitening agent is separated from the aqueous carrier by encapsulation of the whitening agent.

In certain embodiments, the composition comprises about 65%-99.9% of the carrier and further included ingredients, i.e. one or more of anti-caries agents, desensitizing agents, viscosity modifiers, diluents, surfactants, emulsifiers, foam modulators, pH modifying agents, abrasives, mouth feel agents, sweetening agents, flavor agents, colorants, preservatives, amino acids, anti-oxidants, anti-calculus agents, a source of fluoride ions, thickeners, an active agent for prevention or treatment of a condition or disorder of hard or soft tissue of the oral cavity, a whitening agent and combinations thereof. In another embodiment of the composition, the composition comprises about 80%-99.5% of the carrier and further included ingredients. In another embodiment of the composition, the composition comprises about 90%-99% of the carrier and further included ingredients.

In some embodiments, an abrasive polishing material may also be included in the compositions of the present invention. In some embodiments, the abrasive polishing material may be any material which does not excessively abrade dentin. These include, for example, silicas including gels and precipitates, calcium carbonate, dicalcium orthophosphate dihydrate, calcium pyrophosphate, tricalcium phosphate, calcium polymetaphosphate, insoluble sodium polymetaphosphate, hydrated alumina, and resinous abrasive materials such as particulate condensation products of urea and formaldehyde, and others such as disclosed by Cooley et al. in U.S. Pat. No. 3,070,510, Dec. 25, 1962, incorporated herein by reference. Mixtures of abrasives may also be used.

In some embodiments, the abrasive system comprises a silica. Under one embodiment, the silica functions as an abrasive agent. Under another embodiment the silica functions as a thickening agent. Under still another embodiment, the oral care composition comprises both an abrasive silica and a thickening silica.

Silicas suitable for use in the compositions of the present invention may be prepared by any means known or to be developed in the art, and may be surface modified, if desired, to increase the capacity of the particle to adhere to a tooth surface. Examples may be found in, e.g., U.S. Patent Application Publication No. 20070104660, the contents of which are incorporated herein by reference. In the embodiments, silica is present in the composition in an amount of 5% or greater by weight of the total composition. Alternatively, silica may be present in an amount of 5%, 6%, 7%, 8%, 9%, 10%, 15%, 20% or 25% by weight.

In some embodiments, the silica comprises precipitated silica. Precipitated silica is an amorphous form of silica (silicon dioxide, SiO₂), which is a white, powdery material. Precipitated silica is produced by precipitation from a solution containing silicate salts. Under one embodiment, the production of precipitated silica starts with the reaction of an alkaline silicate solution with a mineral acid. Sulfuric acid and sodium silicate solutions are added simultaneously with agitation to water, followed by a precipitation carried out under alkaline conditions. The choice of agitation, duration of precipitation, the addition rate of reactants, their temperature and concentration, and pH can vary the properties of the silica. The formation of a gel stage is avoided by stirring at elevated temperatures. The resulting white precipitate is filtered, washed and dried in the manufacturing process.

Examples of silica include ZEODENT® 105-High, ZEODENT® 103, ZEODENT® 113, ZEODENT® 115, ZEODENT® 116, ZEODENT®117, ZEODENT® 120, ZEODENT® 124, ZEODENT® 153, ZEODENT® 163, ZEODENT® 165, ZEODENT® 167, ZEODENT® 168, ZEODENT® 203, ZEODENT®9175, available from Evonik; SYLODENT® 750 Silica, SYLODENT® 753 Silica, SYLODENT® 756 Silica, SYLOBLANC® 81 Silica, SYLODENT® SM 850C Silica, SYLOBLANC® 82 Silica, SYLODENT® SM 500T Silica, SYLODENT® SM 614T Silica, available from W. R. Grace; Tixosil® 63, Tixosil® 73, Tixosil® SoftClean™, Tixosil® 331, Tixosil® 43, available from Solvay; SORBOSIL AC33, SORBOSIL AC43, SORBOSIL BFG10, SORBOSIL BFG50, SORBOSIL BFG51, SORBOSIL BFG52, SORBOSIL BFG54, SORBOSIL CBT60S, SORBOSIL CBT70, SORBOSIL BFG100, available from PQ Corporation.

In certain embodiments, the silica comprises Sorbosil AC43 silica, available from PQ Corporation. In an embodiment, AC43 silica has properties including, an average particle size of 2.7 to 4.0 microns (as determined by MALVERN MASTERSIZER), a sieve residue of +45 μm, a moisture loss at 105° C. of 8.0% max, an ignition loss at 1000° C. of 14.0% max, and a pH of 5.5 to 7.5 in aqueous suspension.

In some embodiments, the thickener silica is a synthetic amorphous precipitated material of high surface area and internal pore volume to provide water absorption of about 50 ml or greater/20 grams of silica and oil absorption of about 200 ml or greater/100 g silica (per ASTM D281 method). Examples of thickener silicas which may be used are Zeodent® 165, Zeodent® 163 and Zeodent® 153; Aerosil® 200 and Sident® 22S (available from Evonik); Sylodent® 15 and Perkasil® SM 660 (available from W.R. Grace & Co.); MFIL®, MFIL® (available from Madhu Silica, India) and Tixocil 43B (available from Rhodia).

In other embodiments, the oral care compositions of the invention include silica particles with, for example, a particle size distribution of 3 to 4 microns, or alternatively, a particle size distribution of 5 to 7 microns, alternatively, a particle size distribution of 3 to 5 microns, alternatively, a particle size distribution of 2 to 5 microns, or alternatively, a particle size distribution of 2 to 4 microns.

Sodium bicarbonate can also be added to the oral care compositions of the present invention. Sodium bicarbonate, also known as baking soda, is a household product with a variety of uses including use in dentifrices and mouthrinses. It is a white powder that is soluble in water and unless stabilized, tends to release carbon dioxide in an aqueous system.

In some embodiments, the compositions of the present invention comprise a coloring agent. In some embodiments, the coloring agent comprises a pigment. As used herein, a “pigment” is a synthetic or natural water insoluble substance, which imparts color to another substance. In some embodiments, the pigments further enhance the whiteness of the teeth. As is known in the art, the visual perception of a white substance can be altered through the deposition of an optical brightener, a blue pigment, or a blue dye. This effect is commonly used in laundry detergent products to make white clothes appear “whiter” to the human eye. The same concept has been applied to tooth whitening. See PCT Publication No. WO 2015/099642 to Colgate-Palmolive Company, which is herein incorporated by reference in its entirety.

In other embodiments, the pigment is capable of reflecting sufficient light such that the treated tooth is perceivably whiter than its initial color. In some embodiments, the pigment may be colored such that its natural color is within the violet-red to green-blue color. More particularly, the pigment may be violet or blue, e.g., one of those listed in the Color Index International. These pigments are listed as violet pigment #1 through to #56 and blue pigment #1 through #83. In some embodiments, the violet pigment may be violet pigment #1, 1:1, 1:2, 2, 3, 5:1, 13, 19, 23, 25, 27, 31, 32, 37, 39, 42, 44 and/or 50. In some embodiments, the blue pigments may be blue pigment #1, 2, 9, 10, 14, 15, 15:1, 15:2, 15:3, 15:4, 15:6 16, 18, 19, 24:1, 25, 56, 60, 61, 62 and/or 66. Other suitable pigments are pigment ultramarine blue and ultramarine violet. Typically, the pigment is blue pigment #15, more typically blue pigment #15:1, 15:2, 15:3, 15:4, 15:5 or 15:6, most typically 15:1.

In some embodiments, the amount of pigment in the composition may be from 0.01 to 0.075 weight %, such as 0.05%. In other embodiments, the amount of pigment in the composition may be from 0.01 to 0.05 weight %, or from 0.03 to 0.05%, by weight based on the total amount of the composition. The pigment may be uniformly spread throughout the composition or may be dispersed in a second phase such as a stripe or other coextruded second phase. Such “dual phase” compositions have the advantage that the phases may be differently colored, presenting a more visually attractive product to the consumer.

In some embodiments, the coloring agent comprises a dye. As used herein, the term “dye” refers to an organic species, which is essentially water soluble in an aqueous medium in which the dye remains chemically stable. The dyes used with the whitening dentifrice composition of the present disclosure are generally food color additives presently certified under the Food Drug & Cosmetic Act for use in food and ingested drugs, including dyes such as FD&C Red No. 3 (sodium salt of tetraiodofluorescein), FD&C Yellow No. 5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-{[4-(N-ethyl-p-sulfobenzylamino)-phenyl]-(4-hydroxy-2-sulfonium-phenyl)-methylene}-[1-N-ethyl-N-p-sulfobenzyl)-.DELTA.-3,5- cyclohexadienim-ine], FD&C Blue No. 1 (disodium salt of dibenzyldiethyl-diaminotriphenylcarbinol trisulfonic acid anhydride), FD&C Blue No. 2 (sodium salt of disulfonic acid of indigotin) D&C Green No. 5, D&C Orange No. 5, D&C Red No. 21, D&C Red No. 22, D&C Red No. 27, D&C Red No. 28, D&C Red No. 30, D&C Red No. 40, D&C Yellow No. 10 and mixtures thereof in various proportions.

The amount of one or more of the dyes in the oral care composition may widely vary. For example, the amount of one or more of the dyes in the whitening dentifrice composition of the present disclosure may be from 0.02 to 2 weight %, or 0.02 to 1.5 weight %, or 0.02 to 1 weight %, or 0.02 to 0.5 weight %, 0.02 to 0.15 weight %, or 0.02 to 0.1 weight %, based on the total amount of the whitening dentifrice composition. In at least one embodiment, the one or more dyes may be disposed or dispersed uniformly throughout the whitening dentifrice composition. In another embodiment, the one or more dyes may be disposed or dispersed in different phases of the whitening dentifrice composition. For example, one or more of the dyes may be disposed or dispersed in a first phase (e.g., a hydrophobic phase) of the whitening dentifrice composition, and one or more of the remaining dyes, or no dye, may be disposed or dispersed in a second phase (e.g., a hydrophilic phase) of the whitening dentifrice composition.

In some embodiments, the surfactant is selected from water-soluble salts of C₈₋₂₀ alkyl sulfates, sulfonated monoglycerides of C₈₋₂₀ fatty acids, sarcosinates, taurates, sodium lauryl sulfate, sodium cocoyl mono glyceride sulfonate, sodium lauryl sarcosinate, sodium lauryl isoethionate, sodium laureth carboxylate and sodium dodecyl benzenesulfonate, cocoamidopropyl betaine, and mixtures thereof.

Further examples of suitable surfactants include water-soluble salts of higher fatty acid monoglyceride monosulfates, such as the sodium salt of monosulfated monoglyceride of hydrogenated coconut oil fatty acids; higher alkyl sulfates such as sodium lauryl sulfate; alkyl aryl sulfonates such as sodium dodecyl benzene sulfonate; higher alkyl sulfoacetates, such as sodium lauryl sulfoacetate; higher fatty acid esters of 1,2-dihydroxypropane sulfonate; and the substantially saturated higher aliphatic acyl amides of lower aliphatic amino carboxylic compounds, such as those having 12-16 carbons in the fatty acid, alkyl or acyl radicals; and the like. Examples of the last mentioned amides include N-lauryl sarcosine, and the sodium, potassium and ethanolamine salts of N-lauryl, N-myristoyl, or N-palmitoyl sarcosine. Others include, for example, nonanionic polyoxyethylene surfactants, such as Poloxamer 407, Steareth 30, Polysorbate 20, and castor oil; and amphoteric surfactants, such as cocamidopropyl betaine (tegobaine), and cocamidopropyl betaine lauryl glucoside; condensation products of ethylene oxide with various hydrogen containing compounds that are reactive therewith and have long hydrocarbon chains (e.g., aliphatic chains of from 12 to 20 carbon atoms), which condensation products (ethoxamers) contain hydrophilic polyoxyethylene moieties, such as condensation products of poly (ethylene oxide) with fatty acids, fatty, alcohols, fatty amides and other fatty moieties, and with propylene oxide and polypropylene oxides.

In some embodiments, the viscosity modifier is selected from methylcellulose, hydroxypropyl methyl cellulose, hydroxyethylpropyl cellulose, hydroxybutyl methyl cellulose, carboxymethyl cellulose, salts thereof, and mixtures thereof.

In other embodiments, the compositions of the invention may optionally comprise an additional orally acceptable thickening agent, selected from one or more of, without limitation, carbomers, also known as carboxyvinyl polymers, carrageenans, also known as Irish moss and more particularly carrageenan (iota-carrageenan), high molecular weight polyethylene glycols (such as CARBOWAX®, available from The Dow Chemical Company), cellulosic polymers such as hydroxyethylcellulose, carboxymethylcellulose (CMC) and salts thereof, e.g., CMC sodium, natural gums such as karaya, xanthan, gum arabic and tragacanth, and colloidal magnesium aluminum silicate and mixtures of the same. Optionally, such additional thickening agents are present in a total amount of about 0.1 wt % to about 50 wt %, for example about 0.1 wt % to about 35 wt % or about 1 wt % to about 15 wt %, based on the weight of the composition.

In some embodiments, the compositions of the present invention comprise at least one sweetener, useful for example to enhance taste of the composition. Any orally acceptable natural or artificial sweetener can be used, including without limitation dextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, dipeptide-based intense sweeteners, cyclamates and the like.

Still further embodiments provide compositions comprising a sweetener selected from: aspartame; acesulfame potassium; luo han guo (monk) fruit extract; neotame; saccharin; stevia; sucralose; xylitol; advantame; and mixtures thereof.

One or more sweeteners are optionally present in a total amount depending strongly on the particular sweetener(s) selected, but typically 0.005 wt. % to 5 wt. %, by total weight of the composition.

In some embodiments, the composition comprises a fluoride ion source. Fluoride ion sources include, but are not limited to: stannous fluoride, sodium fluoride, potassium fluoride, potassium monofluorophosphate, sodium monofluorophosphate, ammonium monofluorophosphate, sodium fluorosilicate, ammonium fluorosilicate, amine fluoride such as olaflur (N′-octadecyltrimethylendiamine-N,N,N′-tris(2-ethanol)-dihydrofluoride), ammonium fluoride, and combinations thereof. In certain embodiments the fluoride ion source includes stannous fluoride, sodium fluoride, amine fluorides, sodium monofluorophosphate, as well as mixtures thereof. In certain embodiments, the oral care composition of the invention may also contain a source of fluoride ions or fluorine-providing ingredient in amounts sufficient to supply about 50 to about 5000 ppm fluoride ion, e.g., from about 100 to about 1000, from about 200 to about 500, or about 250 ppm fluoride ion. Fluoride ion sources may be added to the compositions of the invention at a level of about 0.001 wt. % to about 10 wt. %, e.g., from about 0.003 wt. % to about 5 wt. %, 0.01 wt. % to about 1 wt., or about 0.05 wt. %. However, it is to be understood that the weights of fluoride salts to provide the appropriate level of fluoride ion will obviously vary based on the weight of the counter ion in the salt, and one of skill in the art may readily determine such amounts.

In some embodiments, the oral care composition is in a form selected from: a toothpaste; a liquid (e.g. a mouthwash or mouthrinse); a gel; a spray; a strip; a powder; a prophy; or a composition which is applied to the teeth using a dental tray. In certain embodiments, the composition is in the form of a toothpaste. In some embodiments, the toothpaste is adapted to be applied to the teeth by brushing. In other embodiments, the oral care composition is in the form of an ingestible or non-ingestible solid (e.g. a tablet or bead).

Embodiments of the present invention will now be further described by way of the following, non-limiting, examples.

### EXAMPLES

### Example 1

Two solutions comprising exemplary complexes of the present invention (Ex. I and Ex. II) and two solutions comprising comparative compositions (Comp. Ex. I and Comp. Ex. II) were prepared. The formulas for these solutions are described below in Table 1.

TABLE 1

| | | | Sodium Lauryl | |
|---|---|---|---|---|
| | | Sorbitol | Sulfate | |
| Example | Tetrahydrocurcurmin | (70% Solution) | (29% Solution) | Water |
| Ex. I | 0.3 g | 4 g | 1.5 g | |
| Comp. Ex. I | 0.3 g | 4 g | | 1.5 g |
| Ex. II | 0.3 g | | 1.5 g | 4 g |
| Comp. Ex. II | 0.3 g | | | 5.5 g |

The four compositions described in Table 1 (above) were aged at 60° C. for two weeks. After two weeks, the tetrahydrocurcumin content in each composition was analyzed using LC-HRMS. Separation was run with a BioBasic C₁₈ LC column from Thermo Fisher (Thermo Scientific, SanJose, USA) and mobile phase, whose composition is acetonitrile/water/NH4OH at 30%/69.8%/0.2%. The flow rate was 150 uL/min, at room temperature. Table 2 (below) describes the tetrahydrocurcumin recovery observed with the compositions described above in Table 1.

TABLE 2

| | Tetrahydrocurcurmin |
|---|---|
| | Recovery |
| Example | (%) |
| Ex. I | 100% |
| Comp. Ex. I | 82.3% |
| Ex. II | 96.4% |
| Comp. Ex. II | 70.2% |

As illustrated by the data described in Table 2 (above), solutions containing exemplary complexes of the present invention provide an unexpected level of tetrahydrocurcumin recovery compared to the solutions that do not contain the exemplary complexes of the claimed invention.

### Example 2

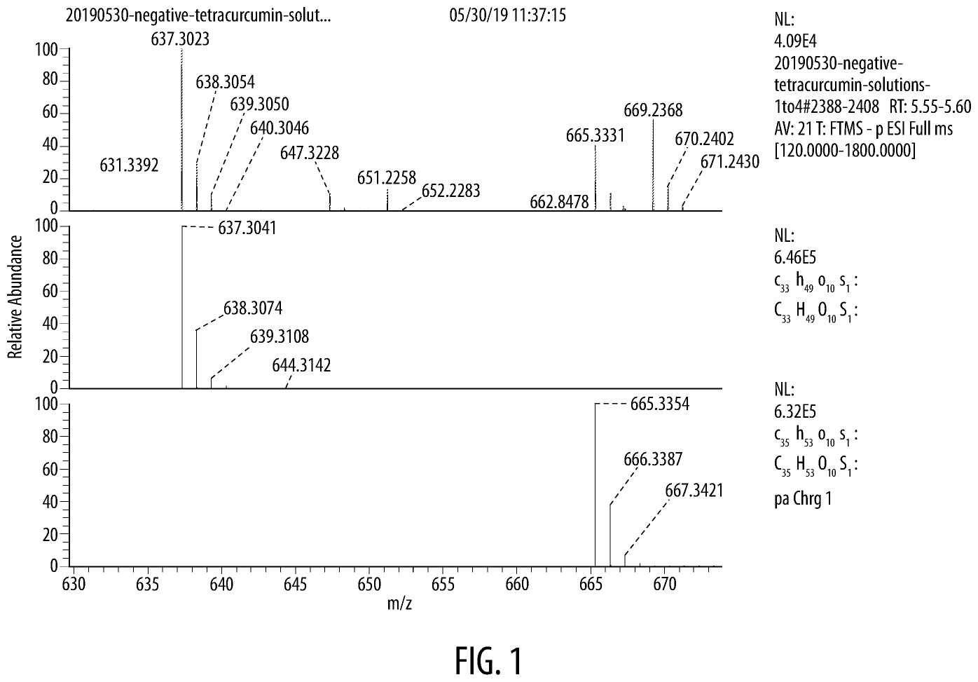

Liquid chromatography mass spectrometry (LCMS) analysis was carried out with a Q-Exactive™ Orbitrap™ mass spectrometer (Thermo Scientific, SanJose, USA) equipped with a heated electrospray ionization source (HESI-II). Samples were analyzed in full scan MS mode under negative polarity condition using electron spray ionization. The compound and source parameters were optimized for both modes for all four solutions. Where as the UHPLC parameters were kept constant. The optimized parameter settings common for both the modes were: sheath and auxiliary gas flow rate at 30 and 10 respectively, spray voltage 3.2 kV, capillary temperature 320° C., S-lens RF level 50, auxiliary gas heater temperature 400° C. Software used for operating the LC-HRMS was Xcalibur™ (version4.1). The results of the LCMS analysis are described in FIG. 1. As demonstrated therein, the complex formed from tetrahydrocurcumin and sodium lauryl sulfate is confirmed by mass spectra, with the high resolution mass spectra simulated from tetrahydrocurcumin plus LS C12 and C14 at 637.3074 (C₃₃H₄₉O₁₀S₁) and 665.3354 (C₃₅H₅₃O₁₀S₁), respectively.

### Example 3

To further evaluate the inventive complexes of the present invention, the diffusion coefficients of a composition comprising an inventive complex of the present invention and a comparative composition which does not include an exemplary complex of the present invention were compared. As demonstrated by the data described in Table 3 (below), the diffusion coefficient of the composition comprising an inventive complex of the present invention was reduced by a factor of five (5) as compared to the diffusion coefficient of the comparative composition which does not include an exemplary complex of the present invention.

TABLE 3

| | Diffusion |
|---|---|
| | Coefficient |
| Example | (m\hat{ }2/s) |
| Ex. III | 1.44E−10 |
| Comp. Ex. III | 7.22E−10 |

### Example 4

Compositions of the present invention can be prepared according to the following method: Step 1: Prepare Surfactant Pre-mix: To a vessel containing 3.7% water, add 1.5% anionic surfactant slowly and mix till fully dispersed.

Step 2: Prepare Gel Pre-mix: To vessel add 63.7% polyol humectant, with mixing add remaining water, then add PEG-600. Mix until completely dispersed. Add sweetener, then fluoride source, and mix for 5 mins till dispersed. Heat to 60-65° C., then slowly add cellulosic material to mixture. Then mix gel for at least about 20 minutes.
Step 3: Prepare Curcuminoid Pre-mix: Add surfactant pre-mix from Step 1 to vessel with remaining polyol humectant. Add curcuminoid and mix until completely dispersed.
Step 4: Add product of Step 3 to product of Step 2 and mix for 5 minutes. Add colorant and mix for 2 minutes.
Step 5: Transfer Gel Phase to Main Mixer. Add abrasive and thickener and mix for 20 minutes under full vacuum. Add flavor, remaining surfactant, and betaine and mix for 10 minutes under full vacuum. Collect product for filling tubes.

### Example 5

Several exemplary compositions of the present invention are described below in Table 4.

TABLE 4

| | Ex. IV | Ex. V | Ex. VI | Ex. VII |
|---|---|---|---|---|
| Ingredient | Wt. % | | | |
| Carrier | 60-75 | 40-50 | 40-50 | 40-50 |
| Fluoride Ion Source | 0.05-0.5 | 0.5-1 | 0.5-1 | 0.5-1 |
| Silica(s) | 10-20 | 0.1-2 | 0.1-2 | 0.1-2 |
| Calcium Abrasives | — | 35-45 | 35-45 | 35-45 |
| Sodium Bicarbonate | — | 0.1-1 | 0.1-1 | 0.1-1 |
| Anionic Surfactant | 0.5-2 | 0.5-3 | 0.5-3 | 0.5-3 |
| Silicate(s) | — | 0.5-2 | 0.5-2 | 0.5-2 |
| Amphoteric Surfactant | 0.5-2 | 0-2 | 0-2 | 0-2 |
| Polyphosphate(s) | — | 0.1-3 | 0.1-3 | 0.1-3 |
| Minors | 0.1-7 | 0.1-7 | 0.1-7 | 0.1-7 |
| (e.g. flavorants, colorants) | | | | |
| Curcuminoid(s) | 0.01-1 | 0.01-1 | 0.01-1 | 0.01-1 |
| Preservative(s) | 0.05-2 | 0.05-2 | 0.05-2 | 0.05-2 |
| Total | 100 | 100 | 100 | 100 |

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

1. An oral care composition comprising:
an orally acceptable carrier; and
a complex comprising:
a curcuminoid; and
an anionic surfactant.

2. The oral care composition according to claim 1, wherein the curcuminoid is selected from: curcumin (tetrahydrodiferuloylmethane); a curcumin derivative; a curcumin analogue; and a preparation of the plant Curcuma longa or another curcumin-containing plant.

3. The oral care composition according to claim 1, wherein the curcuminoid is selected from: curcumin; turmeric; phydroxycinnamoyl(feruloyl)methane, p,p′-dihydroxydicinnamoylmethane, desmethoxycurcumin, bis-desmethoxycurcumin, sodium curcuminate, diacetylcurcumin, triethylcurcumin, and tetrahydrocurcumin.

4. The oral care composition according to claim 1, wherein the curcuminoid comprises tetrahydrocurcumin.

5. The oral care composition according to claim 1, further comprising a polyol humectant.

6. The oral care composition according to claim 5, wherein the polyol humectant is selected from sorbitol; glycerin; and a combination thereof.

7-9. (canceled)

10. The oral care composition according to claim 1, wherein the anionic surfactant comprises an alkyl sulfate surfactant selected from: sodium lauryl sulfate; sodium laureth sulfate; and a combination of thereof.

11-14. (canceled)

15. The oral care composition according to claim 1, wherein the curcuminoid and the anionic surfactant are present in the complex at a molar ratio of 1:1.

16. The oral care composition according to claim 1, wherein at least about 90% of the curcuminoid is recovered after 14 days at 60° C.

17. (canceled)

18. The oral care composition according to claim 1, wherein the complex has a molecular weight of from 637.30 to 665.33.

19-20. (canceled)

21. A conjugate comprising:
a curcuminoid; and
an alkyl sulfate surfactant;
wherein the conjugate has a diffusion coefficient less than the diffusion coefficient of the curcuminoid alone.

22-23. (canceled)

24. The conjugate according to claim 21, wherein the curcuminoid comprises tetrahydrocurcumin.

25. (canceled)

26. The conjugate according to claim 21, comprising tetrahydrocurcumin and sodium lauryl sulfate.

27. The conjugate according to claim 21, wherein the curcuminoid and the alkyl sulfate surfactant are present at a molar ratio of 1:1.

28-31. (canceled)

32. A method of treating, preventing, or ameliorating a symptom associated with, an inflammatory disease, disorder or condition of the oral cavity comprising administering an oral care composition according to claim 1, to the oral cavity of a subject in need thereof.

33-34. (canceled)

35. An oral care composition comprising:
an orally acceptable carrier;
a pre-mix comprising:
a curcuminoid;
a surfactant; and
a polyol humectant.

36. The oral care composition according to claim 35, wherein the pre-mix comprises:
from about 0.01 wt. % to about 1 wt. % of the curcuminoid;
from about 0.5 wt. % to about 5 wt. % of the surfactant; and
from about 1 wt. % to about 10 wt. % of the polyol humectant.

37-39. (canceled)

40. The oral care composition according to claim 35, wherein the curcuminoid is selected from: curcumin; turmeric; phydroxycinnamoyl(feruloyl)methane, p,p′-dihydroxydicinnamoylmethane, desmethoxycurcumin, bis-desmethoxycurcumin, sodium curcuminate, diacetylcurcumin, triethylcurcumin, and tetrahydrocurcumin.

41-42. (canceled)

43. The oral care composition according to claim 35, wherein the surfactant is selected from: sodium cocoyl glutamate; lauryl glucoside; sodium methyl cocoyl taurate; sodium lauroyl sarcosinate; sodium lauryl sulfate; sodium laureth sulfate; cocamidopropyl betaine; sodium cocoamphoacetate; sodium lauryl glucose carboxylate; and a combination of two or more thereof.

44. (canceled)

45. The oral care composition according to claim 35, wherein:
the curcuminoid comprises tetrahydrocurcumin;
the surfactant comprises sodium lauryl sulfate; and
the polyol humectant comprises sorbitol.

46-48. (canceled)
